(19) United States
(12) Patent Application Publication
(10) Pub. No.: US 2009/0036955 A1
(43) Pub. Date: Feb. 5, 2009

(54) Method, System and Apparatus For Guaranteeing Laser Shut-Down Time

(76) Inventor: Xiang Simon Han, Laguna Niguel, CA (US)

Correspondence Address: ALCON, IP LEGAL, TB4-8, 6201 SOUTH FREEWAY, FORT WORTH, TX, 76134, US

(21) Appl. No.: 11/833,291

(22) Filed: Aug. 3, 2007

(51) Int. Cl. H01S 3/00; A61B 18/20

(52) U.S. Cl. 607/89; 372/38.09

(57) ABSTRACT

Systems, methods and apparatuses for guaranteeing a shut-down time for a laser in a laser surgical unit are presented. More particularly, embodiments of such methods, systems and apparatuses may provide shut-down logic which comprises a timer which may be triggered by a signal received from an event. At the expiration of the timer a signal may be output which is configured to disable the output of the laser. Specifically, in one embodiment, software may be used to configure a shut-down time and a set of events to be utilized to start the timer. Upon firing of the laser the timer will be enabled. During the operation of the laser then, if one of the set of events occurs the timer will be triggered such that at the end of the shut-down time the output of the laser will be disabled.

### BACKGROUND OF THE INVENTION

Embodiments described herein relate to surgical devices. More particularly, various embodiments relate to surgical laser systems used in ophthalmic surgical systems. Even more particularly, various embodiments relate to implementations of control or safety measures in conjunction with such a surgical laser systems.

The human eye can suffer a number of maladies causing mild deterioration to complete loss of vision. While contact lenses and eyeglasses can compensate for some ailments, ophthalmic surgery is required for others. Thus, laser surgery to the retina is the standard of care in the treatment of numerous ophthalmic diseases. Diseases treated by laser photocoagulation include proliferative diabetic retinopathy, diabetic macular edema, cystoid macular edema, retinal vein occlusion, choroidal neovascularization, central serous chorioretinopathy, retinal tears, and other lesions.

Generally, ophthalmic surgery is classified into posterior segment procedures, such as vitreoretinal surgery, and anterior segment procedures, such as cataract surgery. More recently, combined anterior and posterior segment procedures have been developed. The surgical instrumentation used for ophthalmic surgery can be specialized for anterior segment procedures or posterior segment procedures or support both. In any case, the surgical instrumentation often implements a whole host of functionality which may be used in the implementation of a wide variety of surgical procedures.

In certain instances, some of this functionality may pose a not insignificant threat of injury to the operator of the device, a person undergoing a surgical procedure or a bystander or observer. For example, a laser utilized in the retinal surgical procedures discussed above may, in certain circumstances, cause injury such as burns, blindness, etc. To avoid circumstances such as these certain safety precautions have been implemented.

For example, the United States Food and Drug Administration (FDA) has mandated that certain laser surgical systems should enter a safe state (e.g. the laser should cease operation or be disabled) within a certain time period (known in the industry as T0) after the occurrence of one or more events. For example, during operation of the laser of the surgical system if a footswitch is disconnected, an active probe removed, a doctor filter disengaged, interlock signal lost, etc. the FDA requires that the laser be shut down or disabled within 50 milliseconds.

Therefore, a need exists for systems, methods or apparatuses to ensure that certain components of a laser surgery unit are placed in a safe state within a certain time period of the occurrence of one or more events.

### SUMMARY

Systems, methods and apparatuses for guaranteeing a shut-down time for a laser in a laser surgical unit are presented. More particularly, embodiments of such methods, systems and apparatuses may provide shut-down logic which comprises a timer which may be triggered by a signal received from an event. At the expiration of the timer a signal may be output which is configured to disable the output of the laser. Specifically, in one embodiment, software may be used to configure a shut-down time and a set of events to be utilized to start the timer. Upon firing of the laser the timer will be enabled. During the operation of the laser then, if one of the set of events occurs the timer will be triggered such that at the end of the shut-down time the output of the laser will be disabled.

Thus, by providing embodiments of these systems, methods and apparatuses the disabling of a laser within a desired time period may be substantially guaranteed. Additionally, embodiments of the systems, methods and apparatuses presented herein may be utilized in conjunction with software such that this software may attempt disable the laser based upon a variety of events (i.e. occurrences or conditions) and if the software does not disable the laser within a desired shut-down time, the laser will still be disabled within the desired time period. Embodiments of the systems, methods and apparatuses may provide the further advantage that they may be configurable to disable the laser based upon the occurrence of one or more of a particular set of events within a certain shut-down time, where this shut-down time may also be configurable.

These, and other, aspects of various embodiments will be better appreciated and understood when considered in conjunction with the following description and the accompanying drawings. The following description, while indicating various embodiments and numerous specific details thereof, is given by way of illustration and not of limitation. Many substitutions, modifications, additions or rearrangements may be made and embodiments can include all such substitutions, modifications, additions or rearrangements.

### BRIEF DESCRIPTION OF THE FIGURES

A more complete understanding of various embodiments and the advantages thereof may be acquired by referring to the following description, taken in conjunction with the accompanying drawings in which like reference numbers indicate like features and wherein:

FIG. 1 is a diagrammatic representation of one embodiment of a laser surgical system;

FIG. 2 is a diagrammatic representation of one embodiment of a laser surgical system coupled to a control unit;

FIG. 3 is a diagrammatic representation of one embodiment of a laser surgical system with shut-down software and shut-down logic; and

FIG. 4 is a diagrammatic representation of one embodiment of shut-down logic.

### DETAILED DESCRIPTION

Preferred embodiments are illustrated in the FIGURES, like numerals being used to refer to like and corresponding parts of the various drawings.

As certain embodiments of the systems methods and apparatuses depicted herein may be utilized in conjunction with a laser surgical unit, it may be helpful to go over embodiments of such a laser surgical unit before discussing embodiments in more detail. Note that these laser surgical consoles are exemplary only and that embodiments of the systems, methods and apparatuses depicted herein may be utilized with other types of devices, laser or otherwise.

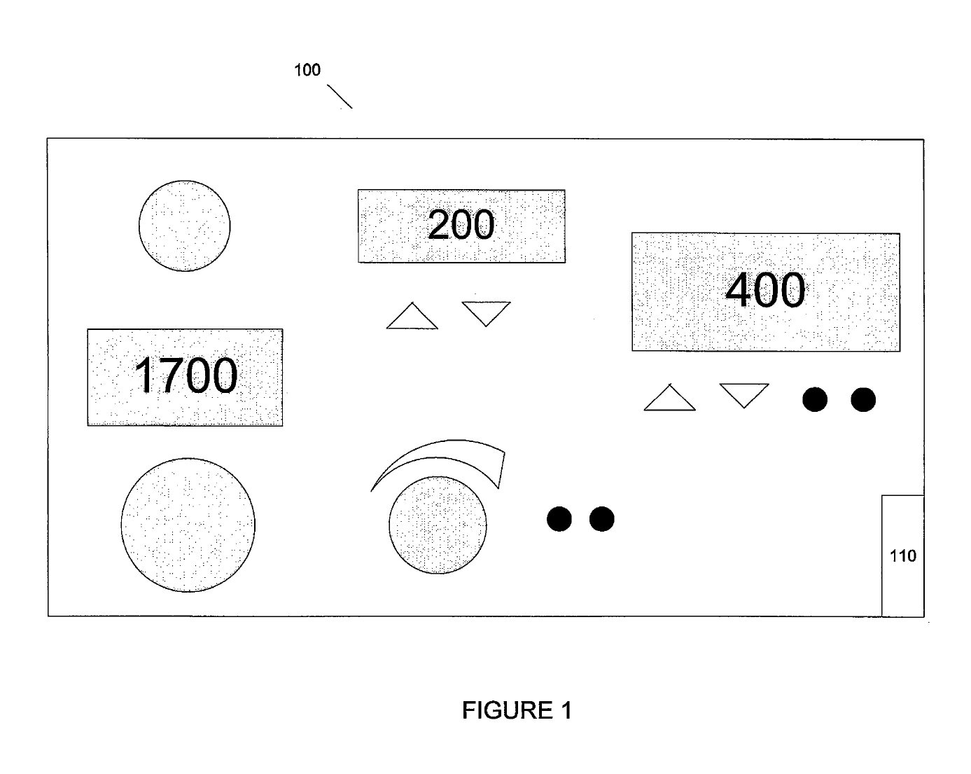

Turning now to FIG. 1, a diagrammatic representation of one embodiment of a laser surgical unit is depicted. Laser surgical unit 100 may comprise a laser engine and associated control hardware and software such that basic laser surgical unit 100 may be operable to implement a set of functionality such as that discussed above. Thus, embodiments of laser surgical unit 100 may provide a lower cost, entry level laser system with a set of functionality particularly well suited to operating room or office use, use in field applications, etc.

In one embodiment, laser surgical unit 100 may have a laser similar to the Alcon 532 Ophthalasa EyeLite Photocoagulator and similar associated software operable to allow the set of functionality to be implemented using laser surgical unit 100. For example, laser surgical unit 100 may utilize an excimer laser. An excimer laser is a type of ultraviolet chemical laser which is commonly used in ophthalmic surgery. An excimer laser typically uses a combination of an inert gas (e.g. Neon) and a reactive gas (e.g. Fluorine). Under appropriate electrical stimulation, laser gas in a laser cavity gives rise to laser light in the ultraviolet range. This light can be focused and is capable of very delicate control. Rather than burning or cutting material, an excimer laser disrupts molecular bonds, thus disintegrating tissue in a controlled manner through ablation rather than burning. Thus excimer lasers have the useful property that they can remove fine layers of tissue with almost no heating or change to surrounding tissue. These properties make excimer lasers well suited for delicate surgeries such as ophthalmic surgery.

To control the functionality or use of such a laser, laser surgical unit 100 may have a variety of control, input, display or coupling devices such as an emergency shut off button, a selector to select an output device for a laser (e.g. a slit lamp, Laser Indirect Ophthalmoscope LIO, endprobe, etc.), a knob to set or control the power of the laser, buttons to set the laser exposure time duration, a button to switch the system between standby and ready mode (e.g. in preparation for firing of the laser), a variety of coupling interfaces operable to couple, for example, a footswitch to control the laser, a probe, a filter for use by a doctor or operator of the surgical unit, etc. Laser surgical unit 100 may also comprise communications port or coupling interface 110, allowing laser surgical unit 100 to be coupled to an another surgical unit such that laser surgical unit 100 may be controlled in conjunction with this other surgical unit.

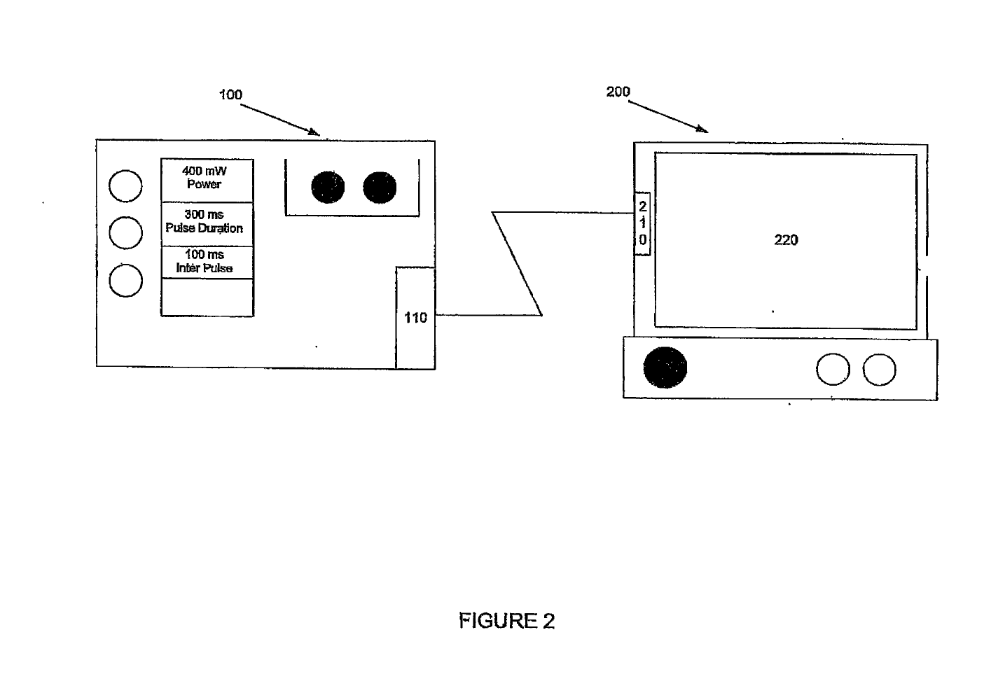

This coupling arrangement may be better described with reference to FIG. 2 which depicts one embodiment of laser surgical unit 100 coupled to a control unit. In one embodiment, laser surgical unit 100 and control unit 200 may be coupled to one another through communications ports 110, 210 on laser surgical unit 100 and control unit 200, respectively. Control unit 200 includes software (e.g. instructions on a computer readable medium), a microprocessor or one or more ASICs, etc. such that advanced control unit 200 is operable to control laser surgical unit 100 or components thereof (e.g. the laser of laser surgical unit 100) to augment the functionality that laser surgical unit 100 may operable to implement in a standalone configuration.

Control unit 200 may be similar to the Series 2000® Legacy® cataract surgical system, the Accurus® 400VS surgical system, the Infiniti™ Vision System surgical system available from Alcon Laboratories Inc. of Fort Worth, Tex. and may also include a connection panel used to connect various tools and consumables to the surgical console. The connection panel can include, for example, a coagulation connector, balanced salt solution receiver, connectors for various hand pieces and a fluid management system (“FMS”) or cassette receiver. A surgical console can also include a variety of user friendly features, such as a foot pedal control (e.g., stored behind a panel) and other features. Advanced control unit 200 may also include swivel monitor 220 which can be positioned in a variety of orientations for whomever needs to see the touch screen of the swivel monitor. Swivel monitor 220 can swing from side to side, as well as rotate and tilt. A graphical user interface (“GUI”) that allows a user to interact with console 100 may be provided or presented on the touch screen of swivel monitor 220.

In some embodiments, the software or microprocessor of control unit 200 may also be operable to implement (e.g. duplicate) the functionality which laser surgical unit 100 is operable to implement in a standalone configuration, such that laser surgical unit 100 can be controlled by advanced control unit 200 in order to implement both this set of functionality and an advanced set of functionality (e.g. a set of functionality which can be implemented utilizing advanced control unit 200 and surgical unit 100 is a superset of the functionality which can be implemented using surgical unit 100 in a standalone configuration). To that end, advanced control unit 200 may also comprise user interface 220, which may, in turn, include a touch screen. This touch screen may serve as an interface through which an operator may select or control the functionality implemented by the combination of advanced control unit 200 and basic laser surgical unit 100.

Thus, a variety of procedures, surgical or otherwise, may be implemented utilizing laser surgical unit 100 (with or without control unit 200). Most if not all of these procedures will involve the laser provided by laser surgical unit 100. As discussed above, the use of these types of lasers may pose a threat of injury to the operator of the device, a person undergoing a surgical procedure or a bystander or observer. Consequently, it may be desired to unilaterally shut down certain functionality of surgical console 100 (e.g. the laser) upon the occurrence of certain events. In fact, to address these concerns certain safety regulations have been imposed by regulatory agencies. For example, the United States Food and Drug Administration (FDA) has mandated that certain laser surgical systems should enter a safe state (e.g. the laser should cease operation or be disabled) within a certain time period (known in the industry as T0) after the occurrence of one or more events. For example, during operation of the laser of the surgical system if a footswitch is disconnected, an active probe removed, incorrect user inputs entered, a doctor filter disengaged, interlock signal lost, etc. the FDA requires that the laser be shut down or disabled within 50 milliseconds.

It is possible to address these eventualities through the use of software. In other words, if software executing on the laser surgical unit 100 (or control unit 200, etc.) detects a shut-down condition the software may disable the laser. However, a variety of conditions may keep this software from accomplishing the disablement of the laser within a desired time period. For example, there may be other software executing and context switching between programs may take longer than expected, an interrupt may be improperly received, there may be a bug in the software, etc. Thus, there may be a need for a method, system or apparatus to guarantee that the laser will be disabled within a certain time period after the occurrence of certain events.

To that end, attention is now directed to methods, systems and apparatuses for guaranteeing a shut-down time for a laser in a laser surgical unit. More particularly, embodiments of such methods, systems and apparatuses may provide shut-down logic (e.g. circuitry, hardware, FPGA, an ASIC, etc.) which comprises a timer which may be triggered by a hardware signal received from an event. At the expiration of the timer a signal may be output which is configured to shut-off or otherwise disable the output of the laser. Specifically, in one embodiment, software may be used to configure a shut-down time (e.g. T0) and a set of events to be utilized to start the timer. Upon firing of the laser the timer will be enabled. During the operation of the laser then, if one of the set of events occurs the timer will be triggered such that at the end of the shut-down time the output of the laser will be disabled.

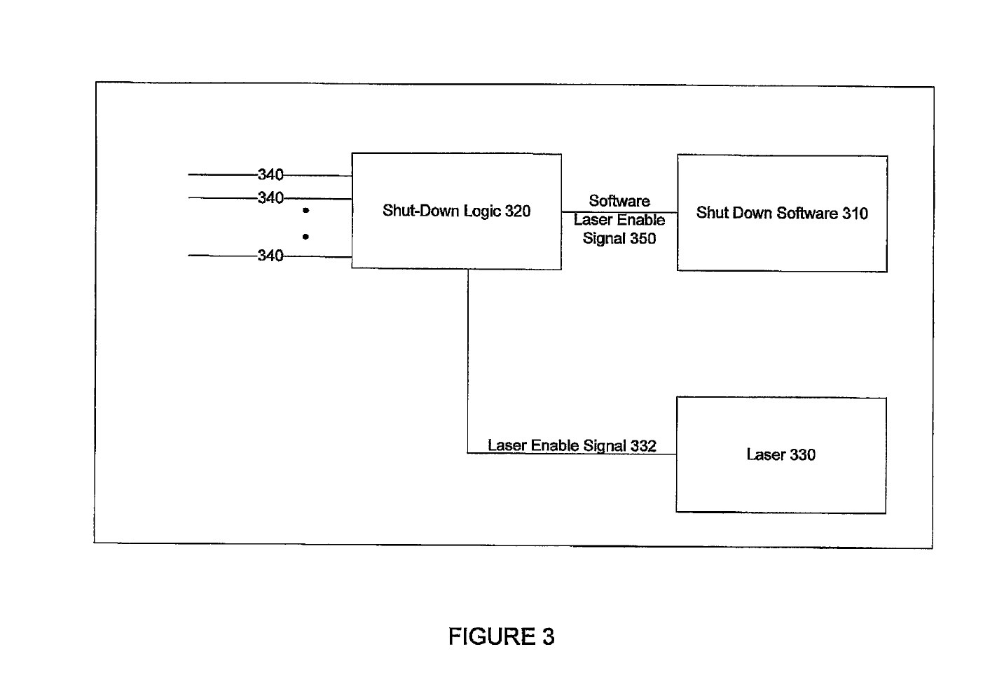

In one embodiment, this shut-down logic may be utilized with shut-down software to provide some degree of fault tolerance regarding the disabling of the laser or to allow the software to disable the laser for a variety of other reasons outside the set of events for which the shut-down logic is configured to utilize. This may be better depicted with reference to FIG. 3 which depicts a diagrammatic representation of a laser surgical unit 300 with shut-down software 310 and shut-down logic 320. A laser enable signal line 332 from shut-down logic 320 may be coupled to laser 330 such that when laser enable signal 332 is low the laser 330 may be disabled. For example, a laser driver (or portions of laser driver circuitry) of laser 330 may be disabled or a shutter may be closed such that the output of laser 330 is cut off, disabling laser 330.

Shut-down logic 320 may be coupled to one or more input signal lines 340 where a change of state on an input signal line may indicate the occurrence of a variety of events. In other words, if one of the input signal lines 340 goes from high to low or low to high some event has occurred (for example, a doctor filter has been unplugged, a probe disconnected etc.). To put it another way, each of the input signal lines 340 may have a firing state (either high or low), indicating that a device, circuitry, register, etc. to which it is coupled is in a safe or desired state for the firing of the laser 330. Whenever this state changes (e.g. the input signal line 340 goes low if the firing state of that input signal line 340 is high or vice versa) an event has occurred. Shut-down logic 320 may therefore create a hardware laser enable signal utilizing the sate of one or more of input signal lines 340.

Shut-down logic 320 may also receive a software laser enable signal 350 from shutdown software 310 indicating that shut-down software 310 has determined that laser 330 should be disabled. Shut-down software 310 may, for example, change the state of software laser enable signal 350 by setting a bit or value of a register or the like, and may change the state of software laser enable signal 350 based upon inputs corresponding to input signal lines 340, or almost any other type of inputs, states, configuration or parameters. Shut-down logic 320 may therefore drive the state of laser enable signal 332 based on the created hardware laser enable signal and the received software laser enable signal 350, in one embodiment by performing a logical AND operation using the state of the hardware laser enable signal and the received software enable signal 350.

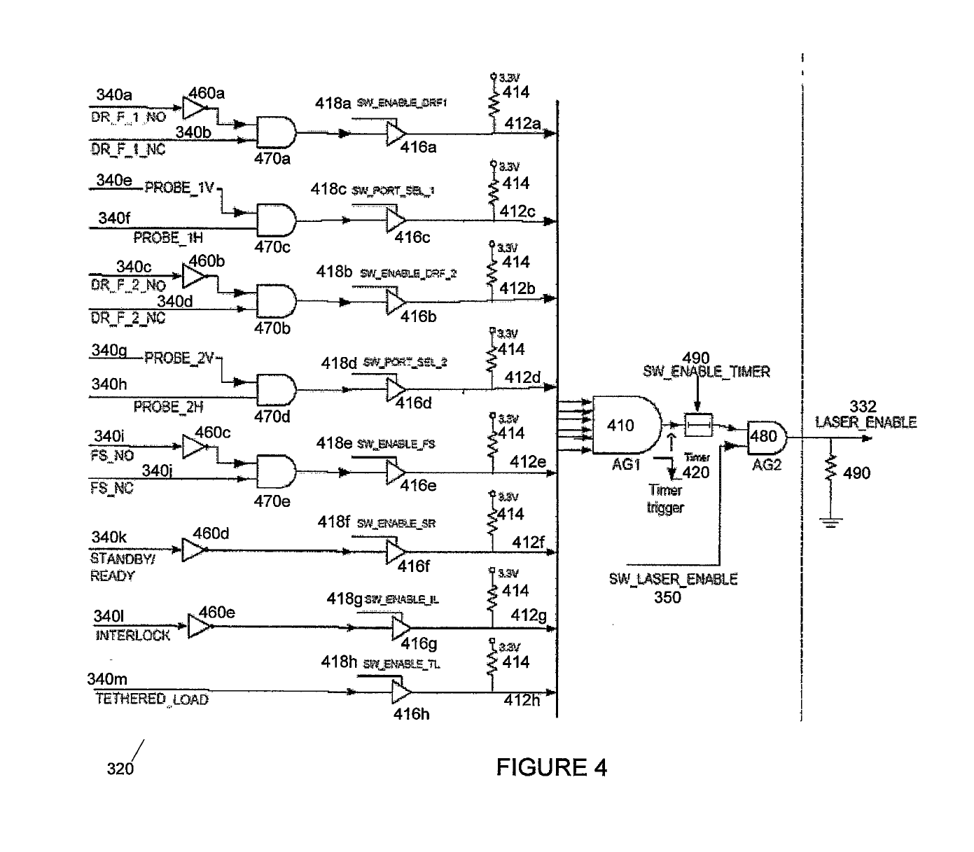

Moving now to FIG. 4, one embodiment of shut-down logic 320 is depicted in more detail. Shut-down hardware 320, which may be a field programmable gate array (FPGA) or the like, comprises AND gate 410 coupled to a plurality of present signal lines 412, each of present signal lines 412 itself coupled to one or more corresponding input signal lines 340. Each of present signal lines 412 is also coupled to a power source (through, for example pull-up resistor 414) and a decoupler 416 (e.g. transistor, capacitor, etc.) such that a present signal line 412 may be decoupled from its one or more corresponding input signal lines 340 based upon a corresponding decoupling signal 418 and, when decoupled, the present signal line 412 is in a high state. If the firing state of an input signal line 340 is low, it may be coupled to an inverter 460 such that the output of the inverter may drive the present signal line 412, while if a present signal line 412 corresponds to multiple input signal lines 340 each of the multiple input signal lines 340 whose firing state is high is coupled to an AND gate 470 and, for each of the multiple input signals 340 whose firing state is low, that input signal line 340 may be coupled to an inverter 460 where the output of the inverter 460 is coupled to the AND gate 470 such that the output of the AND gate 470 may drive a corresponding present signal line 412 based upon each of the multiple input signal lines 340. In other words, when each of the input signals 340 corresponding to a present signal line 412 is in its firing state (e.g. high or low) the present signal line 412 is driven high (if the present signal line 412 is coupled to the input signal lines 340).

It may be helpful here to illustrate in more detail the depicted embodiment of shut-down logic 320. In one embodiment, input signal line 340a labeled “DR_F_1_NO” may be one of two signals from a first doctor filter where the logic level for keeping the laser enabled (i.e. firing state) on input signal line 340a is low (e.g. 0). Input signal line 340b labeled “DR_F_1_NC” may be the second of two signals from the first doctor filter where the firing state is high. Input signal line 340a labeled “DR_F_1_NO” is coupled to inverter 460a the output of which is coupled to the input of AND gate 470a. Another input of AND gate 470a is coupled to input signal line 340b labeled “DR_F_1_NC” while the output of AND gate 470a is coupled to decoupler 416a the output of which is coupled to present signal line 412a and which is controlled by decoupling signal “SW_ENABLE_DRF_1” 418a.

Input signal line 340c labeled “DR_F_2_NO” is one of two signals from a second doctor filter where the firing state is low. Input signal line 340d labeled “DR_F_2_NC” is the second of two signals from the second where the firing state is high. Input signal line 340c labeled “DR_F_2_NO” is coupled to inverter 460b the output of which is coupled the input of AND gate 470b. Another input of AND gate 470b is coupled to input signal line 340d labeled “DR_F_2_NC” while the output of AND gate 470b is coupled to decoupler 416b the output of which is coupled to present signal 412b and which is controlled by decoupling signal “SW_ENABLE_DRF_2” 418b.

Input signal line 340e labeled “PROBE_1V” is one of two signals indicating a laser probe is connected to a first port coupled to the laser, where the firing state is high. Input signal line 340f labeled “PROBE_1H” is the second of two signals indicating a laser probe is connected to the first port coupled to the laser, where the firing state is high. Input signal line 340e labeled “PROBE_V” is coupled to the input of AND gate 470c. Another input of AND gate 470c is coupled to input signal line 340f labeled “PROBEL_1H” while the output of AND gate 470c is coupled to decoupler 416c the output of which is coupled to present signal 412c and which is controlled by decoupling signal “SW_PORT_SEL_1” 418c.

Input signal line 340g labeled “PROBEL_2V” is one of two signals indicating a laser probe is connected to a second port coupled to the laser, where the firing state is high. Input signal line 340h labeled “PROBE_2H” is the second of two signals indicating a laser probe is connected to the second port coupled to the laser where the firing state is high. Input signal line 340g labeled “PROBE_2V” is coupled to the input of AND gate 470d. Another input of AND gate 470d is coupled to input signal line 340h labeled “PROBE_2H” while the output of AND gate 470d is coupled to decoupler 416d the output of which is coupled to present signal 412d and which is controlled by decoupling signal “SW_PORT_SEL_1 ” 418d.

Input signal line 340i labeled “FS_NO” is one of two signals from a footswitch that may be used to turn on the laser where the firing state is low. Input signal line 340j labeled “FS_NC” is the second signal from the footswitch that is used to turn on laser where the firing state is high. Input signal line 340i labeled “FS_NO” is coupled to inverter 460c the output of which is coupled the input of AND gate 470e. Another input of AND gate 470e is coupled to input signal line 340j labeled “FS_NC” while the output of AND gate 470e is coupled to decoupler 416e the output of which is coupled to present signal 412e and which is controlled by decoupling signal “SW_ENABLE_FS” 418e.

Input signal line 340k labeled “STANDBY/READY” may be a signal indicating a system state change between caused by actuation of a Standby and Ready button where the firing state is low. Input signal line 340k labeled “STANDBY/READY” is coupled to inverter 460d the output of which is coupled to decoupler 416f coupled to present signal 412f and controlled by decoupling signal “SW_ENABLE_SR” 418f.

Input signal line 340l labeled “INTERLOCK” may be a signal indicating the status of a lock on the room in which the laser is being utilized where the firing state is low. Input signal line 340l labeled “INTERLOCK” is coupled to inverter 460e the output of which is coupled to decoupler 416g coupled to present signal 412g and controlled by decopling signal “SW_ENABLE_IL” 418g.

Input signal line 340m labeled TETHERED_LOAD may be a signal from an external controller that is coupled to, or controls, the laser unit (as discussed above) where the firing state may be high. Input signal line 340m labeled TETHERED_LOAD may be coupled to decoupler 416h coupled to present signal 412h and controlled by decoupling signal “SW_ENABLE_TL” 418h.

Based upon the state of each of present signal lines 412, AND gate 410 may produce input to timer 420 which may be, in turn, coupled to AND gate 480. Another input of AND gate 480 may be coupled to software laser enable signal 350 such that the output of AND gate 480 drives laser enable signal line 332. During operation of the laser when each of present signals 412 is high the output of AND gate 410 will similarly be high causing output of timer 420 to AND gate 480 to be high, meaning that as long as software laser enable signal 350 is also high, laser enable signal 332 will be high, enabling operation of the laser 330. If, however, during operation of the laser 330 an event occurs which causes an input signal line 340 to change (e.g. from its firing state) the present signal 412 corresponding to that input signal line 340 will go low causing the output of AND gate 410 to similarly go low. When the output of AND gate 410 goes low the falling edge of the output causes timer 420 to start. At the end of a shut-down value associated with timer 420 the output of timer 420 to AND gate 480 will go low, causing the output of AND gate 480 to go low which, in turn, causes output of laser enable signal 332 to go low (which may, in one embodiment, be ensured by the coupling of laser enable signal to pull down resistor 490), disabling laser 330.

In one embodiment, the functioning of shut-down logic 320 may be configured by a user utilizing software (e.g. executing on laser unit 100 or control unit 200) or based upon a system configuration (e.g. whether laser unit 100 is coupled to control unit 200 or used as a standalone device). This configuration may include a user selection of which events should (or should not be) be utilized to control laser 330. These configuration parameters may, for example, be written to registers of, or coupled to, hardware shut-down logic 320. Based on these values, one or more control signals 418 may be asserted causing the corresponding present lines 412 to be decoupled from their corresponding input signal lines 340, where those input signal lines 340 correspond to those events which it desired to disregard or not take into account. In this way any events associated with these input signal lines 340 may be irrelevant the operation (or disabling) of laser 330. For example, suppose a user knows that a second doctor filter and a second probe will not be utilized during a surgical operation. The user may configure laser unit 100 accordingly using software such that this configuration is written to a register. Based on this register then, during operation of laser 330 “SW_ENABLE_DRF_2” decoupling signal 418b may be asserted such that input signal line 340c labeled “DR_F_2_NO” and input signal line 340d labeled “DR_F_2_NC” from a second doctor filter may be decoupled from present line 412b, thus having no effect on the operation of laser 330. Additionally, “SW_PORT_SEL_2” signal 418d may be asserted such that input signal line 340h labeled “PROBE_2H” and input signal line 340g labeled “PROBE_2V” from a second probe may be decoupled from present line 412d, thus having no effect on the operation of laser 330. Similarly, a register reflecting a system configuration (which may be the same or different than the register holding configuration information set by a user) may indicate that laser unit 100 is not coupled to another control unit 200. Here, based upon this register, during operation of laser 330 “SW_ENABLE_TL” signal 418h may be asserted such that input signal line 340m labeled “TETHERED_LOAD” may be decoupled from present line 412h, thus having no effect on the operation of laser 330. In the same manner, a user may configure a shut-down value (e.g. T0) to be utilized with timer 420 during operation of the laser 330. In one embodiment, this shut-down value may be less than a mandated requirement to insure that that the requirement is met. For example, if a mandated shutdown time period is 50 milliseconds a user may configure a shut-down value of 45 milliseconds.

Thus, before, or simultaneous with, activation of laser 330, zero or more decoupling signal lines 418 may be asserted based upon a user or system (or other) configuration indicating which events are not be utilized to control operation of laser 300 effectively decoupling input signal lines 340 corresponding to those events from their corresponding present signal lines 412 (note that the state of present signal line will be high in this case as discussed above). Upon firing or use of laser 330 software enable timer signal 490 may be asserted (e.g. by software) setting the shut-down time (e.g. the configured shut-down time) for timer 420 and causing the output of timer 420 to go high. Software laser enable signal 350 may also be asserted, causing the output of AND gate 480 to go high, driving laser enable signal 332 high, enabling laser 330.

During operation of the laser 330 when each of present signals 412 is high the output of AND gate 410 will be similarly high causing output of timer 420 to AND gate 480 to be high, meaning that as long as software laser enable signal 350 is also high, laser enable signal 332 will be high, enabling operation of the laser 330. If, however, during operation of the laser an event occurs which causes an input signal line 340 which is coupled to a present signal line 412 to change (e.g. from its firing state) the present signal 412 corresponding to that input signal line 340 goes low causing the output of AND gate 410 to similarly go low. When the output of AND gate 410 goes low the falling edge of the output causes timer 420 to start. At the end of the shut-down time associated with timer 420 the output of timer to AND gate 480 will go low, causing the output of AND gate 480 to go low which, in turn, causes output of laser enable signal 332 to go low, disabling laser 330. On the other hand it may be the case that software may detect the occurrence of an event (either the same or a different event) and deassert software laser enable signal 350, causing the output of AND gate 480 to go low which, in turn, causes output of laser enable signal 332 to go low, disabling laser 330 before the expiration of the shut-down value. Thus, shut-down logic 320 may, in one embodiment, shut down laser 330 in cases where software does not respond within the desired shut-down value, providing valuable fault tolerance for software laser shut-down systems.

While various embodiments have been described, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed in the following claims. For example, though embodiments herein have been illustrated in conjunction with certain systems it will be apparent that other embodiments may be utilized in other systems and may be utilized to provide an extra measure of security or safety with respect to the enabling or disabling or various other devices, laser or otherwise. Furthermore, while embodiments have been illustrated to be user configurable and to allow various input signals to be decoupled it will be understood that other embodiments may not have one or more of those capabilities. In addition, while embodiments have been illustrated with respect to certain inputs corresponding to certain events it will be understood that other embodiments may be utilized with almost any desired events or inputs. In the same vein, though certain signal states have been utilized in explaining the embodiments above, it will also be understood that these signal states are exemplary only and that any suitable signal states may be utilized.

What is claimed is:

1. Shut-down logic for use with a laser surgical unit, comprising:
a first AND gate;
a set of present signal lines coupled to the first AND gate;
a timer coupled to an output of the first AND gate;
a laser enable signal, where the a state of the laser enable signal is based upon an output of the timer; and
a laser coupled to the laser enable signal, wherein the laser is configured to be disabled based upon a state of the laser enable signal.

2. The logic of claim 1, wherein each of the present signal lines is coupled to one or more corresponding input signal lines.

3. The logic of claim 2, wherein each of the corresponding input signal lines is coupled to a second AND gate if there is more than one corresponding input signal line, wherein the state of the corresponding present signal line coupled to the output of the second AND gate.

4. The logic of claim 3, wherein a corresponding input signal line is coupled to an input of an inverter if a firing state corresponding to the input signal line is low.

5. The logic of claim 4, wherein the output of the inverter is coupled to the second AND gate if there is more than one input signal line corresponding to the present signal line to which the input signal corresponds.

6. The logic of claim 5, wherein each of the set of input signal lines is associated with an event associated with the operation of the laser surgical unit.

7. The logic of claim 5, wherein the timer is operable to be enabled based upon the output of the first AND gate.

8. The logic of claim 7, wherein each of the present signal lines is coupled to a corresponding decoupler, each decoupler operable to decouple the corresponding set of input lines from the corresponding present signal line based upon a decoupling signal.

9. The logic of claim 8, wherein a state of each of the decoupling signals is based upon a configuration determined in conjunction with software executing on the laser surgical unit.

10. The logic of claim 9, wherein each of the present signal lines is coupled to a power source.

11. The logic of claim 7, wherein an output of the timer is coupled to a third AND gate, a software laser enable signal is coupled to the third AND gate and the output of the third AND gate determines the state of the laser enable signal.

12. The logic of claim 11, wherein a state of the software laser enable signal is determined in conjunction with the software executing on the laser surgical unit.

13. A laser surgical unit, comprising:
a laser; and
shut-down logic, the shut-down logic comprising
a first AND gate;
a set of present signal lines coupled to the first AND gate;
a timer coupled to an output of the first AND gate;
a laser enable signal, wherein a state of the laser enable signal is based upon an output of the timer, wherein the laser is configured to be disabled based upon a state of the laser enable signal.

14. The laser surgical unit of claim 13, wherein each of the present signal lines is coupled to one or more corresponding input signal lines.

15. The laser surgical unit of claim 14, wherein each of the corresponding input signal lines is coupled to a second AND gate if there is more than one corresponding input signal line, wherein the state of the corresponding present signal line coupled to the output of the second AND gate.

16. The laser surgical unit of claim 15, wherein a corresponding input signal line is coupled to an input of an inverter if a firing state corresponding to the input signal line is low.

17. The laser surgical unit of claim 16, wherein the output of the inverter is coupled to the second AND gate if there is more than one input signal line corresponding to the present signal line to which the input signal corresponds.

18. The laser surgical unit of claim 17, wherein each of the set of input signal lines is associated with an event associated with the operation of the laser surgical unit.

19. The laser surgical unit of claim 17, wherein the timer is operable to be enabled based upon the output of the first AND gate.

20. The laser surgical unit of claim 19, wherein each of the present signal lines is coupled to a corresponding decoupler, each decoupler operable to decouple the corresponding set of input lines from the corresponding present signal line based upon a decoupling signal.

21. The laser surgical unit of claim 20, wherein a state of each of the decoupling signals is based upon a configuration determined in conjunction with software executing on the laser surgical unit.

22. The laser surgical unit of claim 21, wherein each of the present signal lines is coupled to a power source.

23. The laser surgical unit of claim 19, wherein an output of the timer is coupled to a third AND gate, a software laser enable signal is coupled to the third AND gate and the output of the third AND gate determines the state of the laser enable signal.

24. The laser surgical unit of claim 23, wherein a state of the software laser enable signal is determined in conjunction with the software executing on the laser surgical unit.

25. A laser surgical unit, comprising:
a first present signal line corresponding to a first doctor filter;
a second present signal line corresponding to a second doctor filter;
a third present signal line corresponding to a first probe;
a fourth present signal line corresponding to a second probe;
a fifth present signal line corresponding to a footswitch;
a sixth present signal line corresponding to a standby/ready button;
a seventh present signal line corresponding to a room lock;
a eighth present signal line corresponding to an external controller;
a first AND gate, where the input of the first AND gate is coupled to the first present signal line, the second present signal line, the third present signal line, the fourth present signal line, the fifth present signal line, the sixth present signal line; the seventh present signal line and the eighth present signal line;
a timer coupled to the output of the first AND gate;
a second AND gate coupled to the output of the timer;
a laser enable signal coupled to the output of the second AND gate, wherein the laser is configured to be disabled based upon a state of the laser enable signal.
